(19) United States
(12) Patent Application Publication
(10) Pub. No.: US 2010/0192239 A1
(43) Pub. Date: Jul. 29, 2010

(54) Modulation of Mu Delta Opioid Receptor Expression

(75) Inventors: Sarah M. Sweitzer, Columbia, SC (US); Steven P. Wilson, New Castle, PA (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/695,676

(22) Filed: Jan. 28, 2010

Related U.S. Application Data
(60) Provisional application No. 61/206,208, filed on Jan. 28, 2009.

(51) Int. Cl. A01K 67/00; C07H 21/04; C12N 15/63; C12N 5/10; C12P 21/06

(52) U.S. Cl. 800/13; 536/23.1; 435/320.1; 435/325; 435/69.1

(57) ABSTRACT

In accordance with certain embodiments of the present disclosure, an expression cassette is provided. The expression cassette includes a heterologous nucleotide sequence that encodes a mu opioid receptor.

### CROSS-REFERENCE TO RELATED APPLICATION

The present application is based on and claims priority to U.S. Provisional Application 61/206,208 having a filing date of Jan. 28, 2009, which is incorporated by reference herein.

### GOVERNMENT SUPPORT CLAUSE

This invention was made with government support under R01 NS026363 awarded by National Institutes of Health. The government has certain rights in the invention.

### BACKGROUND OF THE INVENTION

Physical pain is a highly subjective condition that has been defined as “whatever the experiencing person says it is, existing whenever he says it does.” For scientific and clinical purposes, pain has been defined by the International Association for the Study of Pain (IASP) as “an unpleasant sensory and emotional experience associated with actual or potential tissue damage, or described in terms of such damage”. No matter what definition is used, the experience of pain will be highly individualistic, making pain treatment challenging. Moreover, as pain is a major symptom in many medical conditions, pain treatment must often be integrated with treatment of the underlying causal condition, further complicating treatment options. Accordingly, treatment for both acute and chronic pain conditions can be extremely problematic.

Pain is always a psychological state, but it is closely tied to nociception, which is defined as “the neural processes of encoding and processing noxious stimuli.” Though activity induced in the nociceptor and nociceptive pathways by a noxious stimulus, i.e., a stimulus that is damaging to normal tissues, is not pain, it is often a proximate physical cause of pain, and thus provides a focus for development of pain treatment.

In mammals, the first step in transmission of information about detection of noxious stimuli (nociception) involves sensory or primary afferent neurons. These sensory neurons are bipolar, having axons that extend to their receptive fields in the periphery, e.g., skin, and axons that enter the central nervous system and synapse on second-order neurons in the dorsal horn of the spinal cord. The cell bodies of these neurons reside in ganglia that are located within the spinal column known as spinal or dorsal root ganglia (DRG). Hence, another name for these sensory neurons is dorsal root ganglia neurons.

These DRG neurons are one of several targets for the actions of the opioid drugs, such as morphine, in modifying nociception. Although the predominant site of opioid action is in the brain, receptors for naturally occurring opioid peptides (to which opioid drugs bind) are also found in the peripheral nervous system. The mu opioid receptor (MOR), the primary target for morphine and related compounds, is found in neurons located in the dorsal horn of the spinal cord, both on the presynaptic terminals of DRG neurons and on postsynaptic neurons. Interneurons within the dorsal horn of the spinal cord and possibly neurons descending from the brain release opioid peptides as part of an endogenous, pain-suppressing control mechanism. Systemic administration of mu opioid agonists that do not cross the blood-brain barrier inhibits nociception, demonstrating that activation of peripheral MORs is antinociceptive. At least part of this antinociceptive activity is mediated through MORs on DRG neurons.

What are needed in the art are methods that can exploit endogenous mechanisms regulating nociception to control delivery of antinociceptive therapeutic molecules. In particular, over-expression of MORs would be particularly beneficial.

### SUMMARY

In accordance with certain embodiments of the present disclosure, an expression cassette is provided. The expression cassette includes a heterologous nucleotide sequence that encodes a mu opioid receptor.

In accordance with still other aspects of the present disclosure, a method for over-expression of a mu opioid receptor in a cell is provided. The method includes introducing an expression cassette into the cell in an amount sufficient to over-express the mu opioid receptor. The expression cassette includes a heterologous nucleotide sequence that encodes a mu opioid receptor, wherein the expression cassette over-expresses the mu opioid receptor.

### BRIEF DESCRIPTION OF THE FIGURES

A full and enabling disclosure of the present subject matter, including the best mode thereof, to one of ordinary skill in the art, is set forth more particularly in the remainder of the specification, including reference to the accompanying figures in which:

FIGS. 1 and 2 illustrate effects of disclosed methods on acute neuropathic pain in a rodent model; Herpes viral vectors encoding mu opioid receptor cDNA (SGMOR), decreased mechanical and thermal basal nociception four weeks after topical hind paw infection. A. SGMOR virus decreased basal mechanical nociception, in comparison to the control group. B. SGMOR virus decreased basal thermal nociception, in comparison to control viruses *,***P<05, 0.001 versus control. FIG. 2. HSV-mediated expression of MOR decreased capsaicin-induced spontaneous nociceptive behaviors and thermal hyperalgesia at 4 weeks after topical hindpaw infection. A. SGMOR virus lowered capsaicin-induced spontaneous pain scores five minutes post subcutaneous capsaicin administration in the left hindpaw, in comparison to control viruses. Pain score was calculated based on the equation. [2(time mice spent in licking/flicking of the paw)+time mice spent holding the paw]/60.n, where n=recorded time in minute. B. Ten minutes post capsaicin administration, SGMOR virus attenuated capsaicin-induced thermal hyperalgesia compared to control viruses. *,**,***P<0.05, 0.01, 0.001 compared to control;

FIG. 3 illustrates effects of disclosed methods on chronic neuropathic pain in a rodent model; HSV-mediated decreased expression of MOR decreased neuropathic pain associated mechanical allodynia and thermal hyperalgesia and increased peripheral and systemic opioid analgesia; A. Neuropathic pain was produced by transection of the L5 spinal nerve in mice; B. Mechanical allodynia was measured by eliciting hindpaw withdrawal with von Frey filaments; C. Thermal hyperalgesia was measured by eliciting hindpaw withdrawal with radiant heat; D. On day 7 post-L5 spinal nerve transaction all animals displayed mechanical allodynia as demonstrated by a decrease in the % paw withdrawal threshold as compared to day 0, pre-nerve injury values. Administration of the SGMOR virus on day 7 post-L5 transection reversed mechanical allodynia across the following 2 weeks. E. L5 spinal nerve transaction produced thermal hyperalgesia as demonstrated by a decrease in the % paw withdrawal latency as compared to day 0, pre-nerve injury values. Administration of the SGMOR virus on day 7 post-L5 transection reversed thermal hyperalgesia across the following 2 weeks. F. On day 16 post-SGMOR administration a dose response curve to the peripherally acting opioid agonist loperamide was generated. SGMOR produced a right-ward shift in the dose-response curve suggesting enhanced opioid analgesia. G. On day 16 post-SGMOR administration a dose response curve to the systemically acting opioid agonist morphine was generated. SGMOR produced a right-ward shift in the dose-response curve suggesting enhanced opioid analgesia *,**,***P<0.05, 0.01, 0.001 compared to control; and

FIG. 4 illustrates one embodiment of a shuttle plasmid as may be utilized in disclosed methods.

### DETAILED DESCRIPTION OF REPRESENTATIVE EMBODIMENTS

Reference will now be made in detail to embodiments of the disclosed subject matter, one or more examples of which are set forth below. Each example is provided by way of explanation of the subject matter, not limitation of the subject matter. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

### DEFINITIONS

For purposes of the present disclosure, the term “polypeptide” generally refers to a molecular chain of amino acids and does not refer to a specific length of the product. Thus, peptides, oligopeptides and proteins are included within the definition of polypeptide. This term is also intended to include polypeptides that have been subjected to post-expression modifications such as, for example, glycosylations, acetylations, phosphorylations and the like.

For purposes of the present disclosure, the term “protein” generally refers to any molecular chain of amino acids that is capable of interacting structurally, enzymatically or otherwise with other proteins, polypeptides or any other organic or inorganic molecule.

For purposes of the present disclosure, the term “fragment” in reference to a protein or polypeptide generally refers to an amino acid sequence that is shorter than an entire protein, but comprising at least about 25 consecutive amino acids of the full protein.

For purposes of the present disclosure, the term “ortholog” generally refers to a nucleotide or polypeptide sequence with similar function to a nucleotide or polypeptide sequence in an evolutionarily related species. Loci in two species are said to be “orthologous” when they have arisen from the same locus of their common ancestor. Orthologous polynucleotide sequences exist at loci in different species that are sufficiently similar to each other in their nucleotide sequences to suggest that they originated from a common ancestral sequence. Orthologous sequences arise when a lineage splits into two species, rather than when a sequence is duplicated within a genome. Proteins that are orthologs of each other are encoded by genes of two different species, and the genes are said to be orthologous.

For purposes of the present disclosure, the term “mutant” generally refers to a polypeptide that includes any change in the amino acid sequence relative to the amino acid sequence of the reference polypeptide. Such changes can arise either spontaneously or by manipulations including those chemical derivatives brought about by chemical energy (e.g., X-ray), other forms of chemical mutagenesis, by genetic engineering, or as a result of mating or other forms of exchange of genetic information. Mutations include, e.g., base changes, deletions, insertions, inversions, translocations, or duplications. Mutants may or may not also comprise additional amino acids derived from the process of cloning, e.g., amino acid residues or amino acid sequences corresponding to full or partial linker sequences.

For purposes of the present disclosure, the term “homolog” generally refers to two nucleotide or polypeptide sequences that differ from each other by substitutions that do not effect the overall functioning of the polypeptide. For example, when considering polypeptide sequences, homologs include polypeptides having substitution of one amino acid at a given position in the sequence for another amino acid of the same class (e.g., amino acids that share characteristics of hydrophobicity, charge, pK or other conformational or chemical properties, e.g., valine for leucine, arginine for lysine). Homologs also include polypeptides and nucleotide sequences including one or more substitutions, deletions, or insertions, located at positions of the sequence that do not alter the conformation or folding of the polypeptide to the extent that the biological activity of the polypeptide is destroyed. Examples of possible homologs include polypeptide sequences including substitution of one non-polar (hydrophobic) residue such as isoleucine, valine, leucine or methionine for one another; the substitution of one polar (hydrophilic) residue for another such as between arginine and lysine, between glutamine and asparagine, or between threonine and serine; the substitution of one basic residue such as lysine, arginine or histidine for another; the substitution of one acidic residue, such as aspartic acid or glutamic acid for the another; or the use of a chemically derivatized residue in place of a non-derivatized residue, as long as the homolog polypeptide displays substantially similar biological activity to the reference polypeptide.

For purposes of the present disclosure, the term “analog” generally refers to a non-natural molecule substantially similar to either the entire reference protein or polypeptide, or a fragment or allelic variant thereof, and having substantially the same or superior biological activity. The term “analog” is intended to include derivatives (e.g., chemical derivatives) of the biologically active polypeptide, as well as its fragments, mutants, homologs, orthologs, and allelic variants, which derivatives exhibit a qualitatively similar agonist or antagonist effect to that of the unmodified polypeptide.

For purposes of the present disclosure, the term “allele” generally refers to a polypeptide sequence containing a naturally-occurring sequence variation relative to the polypeptide sequence of the reference polypeptide. Similarly, an allele of a polynucleotide encoding a polypeptide is herein defined to be a polynucleotide containing a sequence variation relative to the reference polynucleotide sequence encoding the reference polypeptide, where the allele of the polynucleotide encoding the polypeptide encodes an allelic form of the polypeptide.

For purposes of the present disclosure, the term “operably linked” generally refers to a situation wherein the components described are in a relationship permitting them to function in their intended manner. For instance, a control sequence “operably linked” to a coding sequence is ligated in such a manner that expression of the coding sequence is achieved under conditions compatible with the control sequence. A “coding sequence” is a polynucleotide sequence which is transcribed into mRNA and translated into a polypeptide when placed under the control of (e.g., operably linked to) appropriate regulatory sequences. The boundaries of the coding sequence are determined by a translation start codon at the 5′-terminus and a translation stop codon at the 3′-terminus. Such boundaries can be naturally-occurring, or can be introduced into or added to the polynucleotide sequence by methods known in the art. A coding sequence can include, but is not limited to, genomic DNA, mRNA, cDNA, and recombinant polynucleotide sequences.

For purposes of the present disclosure, the term “sequence identity,” generally refers to the subunit sequence similarity between two polymeric molecules. For example, the sequence similarity between two polynucleotides or two polypeptides. When a subunit position in both of the two molecules is occupied by the same monomeric subunit, then they are identical at that position. The identity between two sequences is a direct function of the number of matching or identical positions. For example, if half of the positions in two peptide or protein sequences are identical, then the two sequences are 50% identical. The identity between two sequences is a direct function of the number of matching or identical positions. Thus, if a portion of the reference sequence is deleted in a particular peptide, that deleted section is not counted for purposes of calculating sequence identity. For example, when comparing a first polymer including monomers R₁R₂R₃R₄R₅R₆ with another polymer including monomers R₁R₂R₃R₄R₆, the two polymers have 5 out of 6 positions in common, and therefore would be described as sharing 83.3% sequence identity.

For purposes of the present disclosure, the terms “heterologous nucleic acid,” or “exogenous nucleic acid” generally refer to a nucleic acid that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring nucleic acid. Thus, the terms refer to a nucleic acid segment that is foreign or heterologous to the cell, or normally found within the cell but in a position within the cell or genome where it is not ordinarily found.

For purposes of the present disclosure, the terms “isolated” or “purified” nucleic acid or an “isolated” or “purified” polypeptide generally refer to a nucleic acid or polypeptide that, by the hand of man, exists apart from its native environment and is therefore not a product of nature. An isolated nucleic acid or polypeptide may exist in a partially purified or substantially purified form. An isolated nucleic acid or polypeptide may also exist in a non-native environment such as, for example, a transgenic host cell.

For purposes of the present disclosure, the term “vector” generally refers to a nucleic acid that can transfer nucleic acid segment(s) from one cell to another. A “vector” includes, inter alia, any plasmid, cosmid, phage, virus, or nucleic acid in double or single stranded linear or circular form that may or may not be self transmissible or mobilizable, and that can transform prokaryotic or eukaryotic host either by integration into the cellular genome or by existing extrachromosomally (e.g. autonomous replicating plasmid with an origin of replication). Vectors used in bacterial systems often contain an origin of replication so that the vector may replicate independently of the bacterial chromosome. The term “expression vector” refers to a vector containing an expression cassette.

### DETAILED DESCRIPTION

In general, the present disclosure is directed to methods and materials that can be utilized for pain management. More specifically, disclosed methods and materials can over-express mu opioid receptors (MORs) in dorsal root ganglia neurons as a treatment for acute and chronic pain and to enhance opioid analgesia.

Disclosed methods and materials can be utilized in one embodiment for in vivo acute or chronic pain management, for instance via a gene therapy protocol. Disclosed methods and materials can also be utilized for in vitro applications, for instance to better elucidate pain mechanisms and through that knowledge for the development of improved pain management compounds and strategies.

According to one embodiment, disclosed is a recombinant expression cassette that can be inserted into a host cell such that the heterologous genetic information of the recombinant expression cassette can be expressed in the host cell. A transcriptional cassette generally includes in the 5′-3′ direction of transcription a promoter, a transcriptional and translational initiation region, a nucleic acid sequence for an anti-nociceptive compound (e.g., a sense or antisense cDNA encoding antinociceptive proteins), and a transcriptional and translational termination region functional in the targeted cell. The termination region may be native with the transcriptional initiation region, may be native with the DNA sequence of the anti-nociceptive compound, or may be derived from another source.

An expression cassette can include as promoter an isolated promoter/enhancer sequence for a gene. It is known that the expression of certain genes in neurons, e.g., dorsal root ganglia (DRG) neurons, is substantially up-regulated in response to pain-producing conditions (nerve damage, noxious stimuli) or inflammation. Generally, any suitable promoter may be used in accordance with the present disclosure. Such promoters can regulate the transcription of the operably linked heterologous nucleotide sequences of the cassettes.

While suitable promoters can include sequences to which an RNA polymerase binds, this is not a requirement of the disclosure. For example, promoters of the disclosed DNA constructs may include regions to which other regulatory proteins may bind in addition to regions involved in the control of the protein translation, including coding sequences.

Several different categories of UPI promoters are encompassed by the present disclosure including, without limitation, promoters for neuropeptide precursors, ion channels, receptors, transcription factors, and so forth.

Promoters and probes for promoters as may be utilized in disclosed expression cassettes can include, without limitation, promoters as would be known in the art, or orthologs, homologs, or alleles thereof. A promoter sequence can include a sequence that hybridizes to a probe, the nucleic acid sequence of which consists of a known promoter or the complement thereof. In one embodiment, an expression cassette can include as a promoter a nucleic acid sequence that hybridizes to a probe that consists of a disclosed promoter or a complement thereof under stringent conditions, for instance under low stringent conditions in one embodiment, under moderately stringent conditions in another embodiment, or under highly stringent conditions in another embodiment.

A promoter can be used to drive transcription of a heterologous nucleic acid of the expression cassette that encodes an antinociceptive compound such as, for example, receptors that increase the sensitivity of neurons to endogenous antinociceptive molecules or to antinociceptive drugs.

Nucleotide sequences that can be incorporated into disclosed expression cassettes can include any cDNA or gDNA, mRNA, miRNA, and so forth that encodes an antinociceptive compound in a sense orientation. Specifically, the isolated heterologous nucleotide sequence of disclosed constructs is not limited to cDNA sequences, and the antinociceptive compound-encoding construct may include variations as are known to those of skill in the art including orthologs, homologs, and alleles of the nucleic acid encoding the antinociceptive compound, provided the transcribed protein product may exhibit the same or superior response in a host as the DNA encoded transcription products.

Exemplary antinociceptive compounds as may be incorporated into disclosed expression cassettes are shown in Table 2, below, including GenBank accession numbers. Of course, the listed compounds are exemplary embodiments, only, and other variants, e.g., splice variants, orthologs, homologs, alleles, and the like as are known in the art could alternatively be utilized. According to one embodiment, sequences for antinociceptive compounds can include isolated sequences as described below.

TABLE 2

| | | | Amino Acid | Nucleic Acid |
|---|---|---|---|---|
| | | | Sequence | Sequence |
| | | | Accession | Accession |
| Category | Examples | | No. | No. |
| cDNAs for | Mu opioid | rat | NP_037203.1 | NM_013071.2 |
| antinociceptive | receptor | (Oprm1) | NP_000905.3 | NM_000914.3 |
| receptors | | human | | |
| | | (OPRM1) | | |

Constructs disclosed herein are not limited to these specific sequences, however. For instance, in one embodiment, nucleic acid sequences as may be incorporated in disclosed expression cassettes can include a nucleic acid sequence that hybridizes to a probe, the nucleic acid sequence of which consists of a sequence or the complement of a sequence described above in Table 2. In one embodiment, an expression cassette can include a nucleic acid sequence that hybridized to such a probe under stringent conditions, for instance under low stringent conditions in one embodiment, under moderately stringent conditions in another embodiment, or under highly stringent conditions in another embodiment.

In one embodiment, an expression cassette can include a nucleic acid sequence that encodes a polypeptide having an antinociceptive affect, for instance an amino acid sequence as described above in Table 2 or an active fragment, mutant, ortholog, homolog, analog, or allele thereof.

In addition to a promoter and a sequence encoding an antinociceptive compound in a sense orientation, expression cassettes as described herein can also include suitable operably linked regulatory sequences as are generally known to those of skill in the art. For instance, an isolated DNA construct can include DNA encoding one or more of a suitable translation leader sequence, and polyadenylation and transcription termination sequences. An expression cassette can also include a plurality of restriction sites for insertion of the nucleotide sequence to be under the transcriptional regulation of various control elements. The expression cassette additionally may contain selectable marker genes. Suitable control elements such as splice junctions, polyadenylation signals, etc. may be placed in close proximity to the coding region of the antinociceptive compound if needed to permit proper initiation of transcription and/or correct processing of the primary transcript. Alternatively, the coding region utilized in the expression cassette may contain endogenous leader sequences, splice junctions, intervening sequences, polyadenylation signals, etc., or a combination of both endogenous and exogenous control elements.

In one embodiment, isolated polynucleotides encoding an antinociceptive compound can additionally comprise a polynucleotide linker encoding a peptide. Such linkers are generally known to those of skill in the art and can comprise, for example, at least one additional codon encoding at least one additional amino acid. Typically the linker comprises one to about twenty or thirty amino acids. The polynucleotide linker can be translated along with the disclosed polynucleotides resulting in the expression of the disclosed polypeptides with at least one additional amino acid residue at the amino or carboxyl terminus of the polypeptide.

The presently disclosed subject matter is directed not only to the disclosed expression cassettes, but is also directed to vectors and host cells containing such polynucleotides. Vectors encompassed by the present disclosure include any molecules into which pieces of nucleic acid may be inserted or cloned that can transfer the nucleic acids carried thereby into a host cell. In some embodiments of the present invention, vectors may also bring about the replication and/or expression of the transferred nucleic acid pieces. An exemplary list of suitable vectors can include nucleic acid molecules derived from a plasmid, bacteriophage, or mammalian, virus, or non-viral vectors such as ligand-nucleic acid conjugates, liposomes, or lipid-nucleic acid complexes. The vector may, if desired, be a bi-functional expression vector that may function in multiple hosts.

DNA constructs, including a promoter and DNA encoding an antinociceptive compound as well as one or more sequences functional for the expression, processing and secretion of the mature protein in the transgenic host cell, can be designed so as to move between one or more vectors or plasmids and into the target cell.

In one preferred embodiment, a vector for use as disclosed herein can integrate into the host cell DNA, and in one particular embodiment, can be a viral vector. Viral vectors as may be utilized can include, without limitation, those developed from herpes simplex virus, lentivirus, retrovirus, and so forth. Methods for making a viral recombinant vector useful for inserting disclosed expression cassettes into a host cell can be analogous to the methods disclosed in U.S. Pat. Nos. 4,603,112; 4,769,330; 5,174,993; 5,505,941; 5,338,683; 5,494,807; 4,722,848; E. Paoletti, “Applications of Poxvirus Vectors to Vaccination: An Update,” PNAS USA 93:11349-11353, 1996; Moss, “Genetically Engineered Poxviruses for Recombinant Gene Expression, Vaccination and Safety,” PNAS USA 93:11341-11348, 1996; Roizman, “The Function of Herpes Simplex Virus Genes: A Primer for Genetic Engineering of Novel Vectors,” PNAS USA 93:11307-11302, 1996; Frolov at al., “Alphavirus-Based Expression Vectors: Strategies and Applications,” PNAS USA 93:11371-11377, 1996; Grunhaus et al., “Adenoviruses As Cloning Vectors,” Seminars in Virology 3: 237-252, 1993 and U.S. Pat. Nos. 5,591,639; 5,589,466; 5,580,859; 6,193,980; 6,610,287; 6,613,892; and 6,821,753, all of which are incorporated herein by reference, relating to DNA expression vectors.

In one preferred embodiment, delivery, e.g., in vivo delivery, can be accomplished by use of human herpes simplex virus, type 1 (HSV-1). This neurotrophic virus infects most cell types and is rapidly cleared from the body; however, in DRG neurons the DNA genome of the virus persists in the nucleus of the cell in a non-integrated or episomal form for the life of the individual. Recombinant HSV-1 genomes into which exogenous expression cassettes have been incorporated, for instance by homologous recombination, can be delivered to DRG neurons and delivery can result in expression of the encoded sequences in these neurons.

Alternative to a viral vector, any standard plasmid containing an operably linked promoter and antinociceptive sequences as described herein could be used. Moreover, lentivirus or other replication-defective viruses could also be used as vectors, for instance for tissue culture work for in vitro applications.

In one embodiment, a binary vector can be utilized for a disclosed transformation. Binary vectors can be conveniently utilized for independently introducing to a host cell in an unlinked manner a second heterologous nucleotide sequence that can be either the same or different as the sequence encoding the antinociceptive protein. For instance, a plasmid can be utilized that contains a multiple cloning site. Optionally, the vector can be modified to add a restriction site, for example an NdeI site. Such sites are well known to those of skill in the art.

Vectors and plasmids can optionally include nucleotide sequences encoding one or more selectable markers. For example, sequences encoding selectable markers including fluorescent markers such as GFPs, GUS, S-Tags, His-Tags, and the like can be utilized. Proteins and polypeptides produced according to this particular embodiment can comprise a tag, e.g., a histidine tag motif (His.tag) comprising one or more histidines, in one embodiment about 5-20 histidines. Of course, any tag should not interfere with the desired properties of the product.

Following the cloning of an expression cassette including a promoter sequence and an antinociceptive polynucleotide into a suitable vector, for instance via homologous recombination, gene trapping, or any other suitable method as is generally known to one of ordinary skill in the art, the vector can deliver the expression cassette into an appropriate host cell via transduction or transfection, depending upon the nature of the vector. By “host cell” is meant a cell which has been or can be used as the recipient of transferred nucleic acid by means of a vector. Host cells can exist as single cells, or as a collection, e.g., as a culture, or in a tissue culture, or in a tissue or an organism. Host cells can also be derived from normal or diseased tissue from a multicellular organism, e.g., a mammal. Host cell, as used herein, is intended to include not only the original cell which was transformed with a nucleic acid, but also descendants of such a cell, which still contain the nucleic acid.

An expression cassette may be introduced and expressed in a host cell, for example, in either neuronal or non-neuronal host cells. Examples of host cells include, without limitation, DRG cells, central neurons, glial cells (e.g., ependymal cells), and so forth. Preferably, the recombinant host cell system that is selected processes and post-translationally modifies nascent peptides in a manner desired to produce an antinociceptive compound.

In general, any transfection method as is known in the art can be utilized to transform the host cell with a non-viral vector including an expression cassette as disclosed herein. For example, an expression cassette may be introduced into a host cell by commonly used transformation procedures such as by treatment with calcium chloride, with lithium acetate, by calcium phosphate co-precipitation, by spheroplast fusion, by electroporation, and so forth.

In one embodiment, ballistic transformation methods as are generally known in the art can be utilized. For example, microparticles carrying a DNA construct of the present invention can be utilized for the ballistic transformation of a host cell. In other embodiments, plasmid DNA can be propelled into a host cell without particles. For instance, a DNA construct can be propelled into a host cell to produce a transformed host cell. Any suitable ballistic cell transformation methodology and apparatus can be used. Exemplary apparatus and procedures are disclosed in Sanford et al., U.S. Pat. No. 4,945,050, and in Christou et al., U.S. Pat. No. 5,015,580, both of which are incorporated herein by reference. Examples of microparticles suitable for use in such systems can include those utilizing spheres, for instance gold spheres, of from about 1 to about 5 μm in diameter, and the like. The DNA construct may be deposited on the microparticle by any suitable technique, for instance, by precipitation.

The above described transformation methodologies are exemplary only, and in general, any method of inserting DNA into a host cell as is generally known in the art can be used in forming the transgenic cells.

Methods for introduction of disclosed expression cassettes to a host cell using a viral vector by means of viral infection are also encompassed by the present disclosure. Transduction may be performed ex vivo, in vitro, or in vivo, according to standard methodology. For example in vivo transduction of a host cell can be carried out via subcutaneous injection of a viral DNA vector that expresses the polypeptide into laboratory animals, such as mice.

According to one embodiment, the present disclosure provides an expression cassette as disclosed, or a vector such as a virus comprising such an expression cassette, for use in a method of treatment of the human or animal body, as well as use of such an expression cassette or such a vector in the manufacture of a medicament or composition for use in treatment of the human or animal body. For example, according to one embodiment, a cell containing a construct according to the disclosure, e.g., as a result of introduction of the construct into the cell or an ancestor thereof, may be administered to a subject. For example following in vitro or ex vivo transduction or transfection of a host cell, cells may be cultured or maintained ex vivo and then delivered to a subject, either a subject from which they were obtained (or from which an ancestor was obtained) or a different subject.

Alternatively, a vector can be directly administered to an individual. For instance, the administration may be by infection with a viral vector which comprises the construct. Naked DNA delivery may be used. For example, stereotactic injection of the therapeutic virus into the nervous system is an accepted, efficient and widely used procedure for introducing substances to, or biopsying from, specific regions of the central nervous system in both humans and animals.

Administration to an individual subject is preferably in a “therapeutically effective amount,” this being sufficient to show benefit to a subject. Such benefit may be at least amelioration of at least one symptom. The actual amount administered, and rate and time-course of administration, can depend on the nature and severity of pain in a subject. Prescription of treatment, e.g., decisions on dosage etc, is within the responsibility of general practitioners and other medical doctors.

A pharmaceutical composition may be administered alone or in combination with other treatments, either simultaneously or sequentially dependent upon the condition to be treated. Pharmaceutical compositions for use in accordance with the present disclosure, may comprise, in addition to an expression cassette, for instance carried by a viral vector, a pharmaceutically acceptable excipient, carrier, buffer, stabiliser or other materials well known to those skilled in the art. Such materials should be non-toxic and should not interfere with the efficacy of the expression cassette. The precise nature of the carrier or other material will depend on the route of administration, which may be oral, or by injection, e.g. cutaneous, subcutaneous or intravenous. Osmotic minipumps may also be used to provide controlled delivery of high concentrations of materials through cannulae to the delivery site.

For intravascular, cutaneous, subcutaneous, intramuscular, intraocular or intracranial injection, or direct injection into cerebrospinal fluid, injection into the biliary tree, or injection at the site of affliction, the pharmaceutical composition will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Prolonged absorption of an injectable pharmaceutical form may be brought about by the inclusion of agents, such as aluminum monostearate and gelatin, which can delay absorption. For example, injectable depot forms can be made by forming microencapsule matrices including the disclosed expression cassettes, e.g., in deliverable vectors or transformed host cells, in biodegradable polymers such as polylactide-polyglycolide, poly(orthoesters) and poly(anhydrides). Depending upon the ratio of material to polymer and the nature of the particular polymer employed, the rate of release can be controlled. Depot injectable formulations can also be prepared by entrapping the materials in liposomes or microemulsions which are compatible with body tissues.

Reference now will be made to exemplary embodiments of the invention set forth below. Each example is provided by way of explanation of the invention, not as a limitation of the invention.

### Example 1

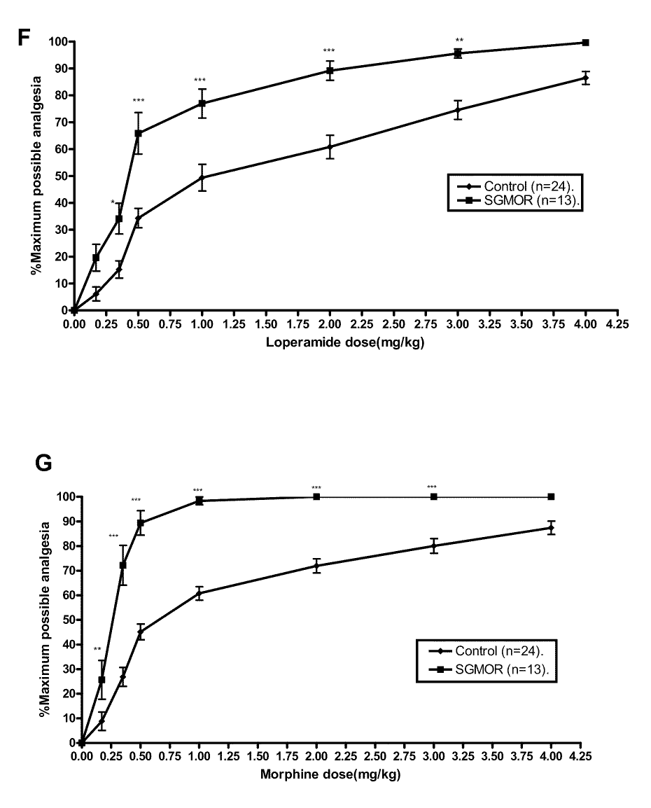

Recombinant herpes viral vectors containing a bicistronic expression cassette composed of the human cytomegalovirus immediate-early promoter-enhancer, the rat MOR cDNA, an internal ribosome entry site (IRES), the gene for enhanced green fluorescent protein (EGFP) and a polyadenylation sequence (PA) were inserted into the region between the viral UL36 and UL37 genes (FIG. 4). High titer stocks of the viruses were then produced and tested in animal models of both acute and chronic pain.

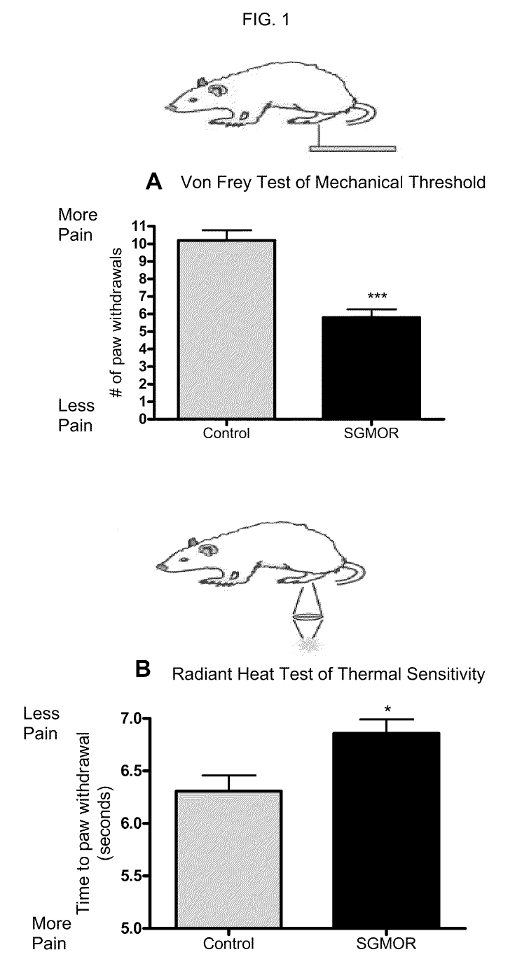

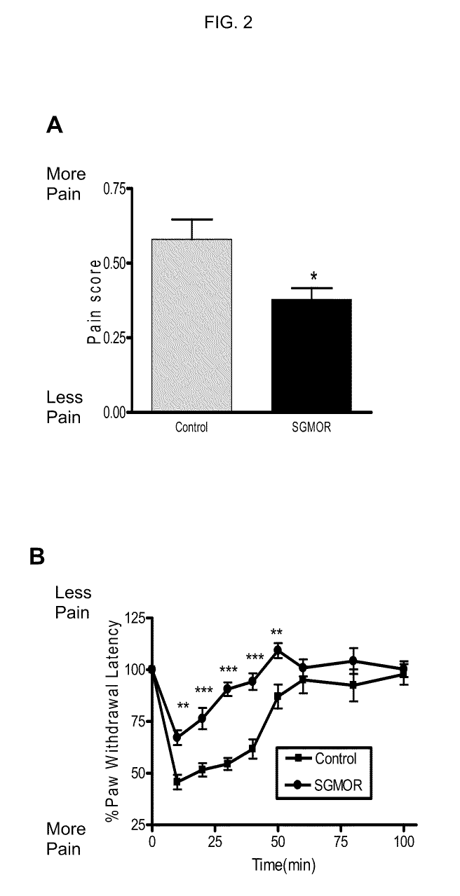

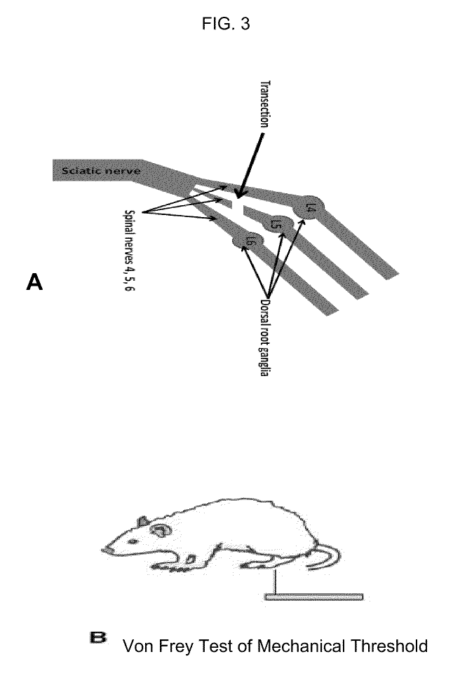

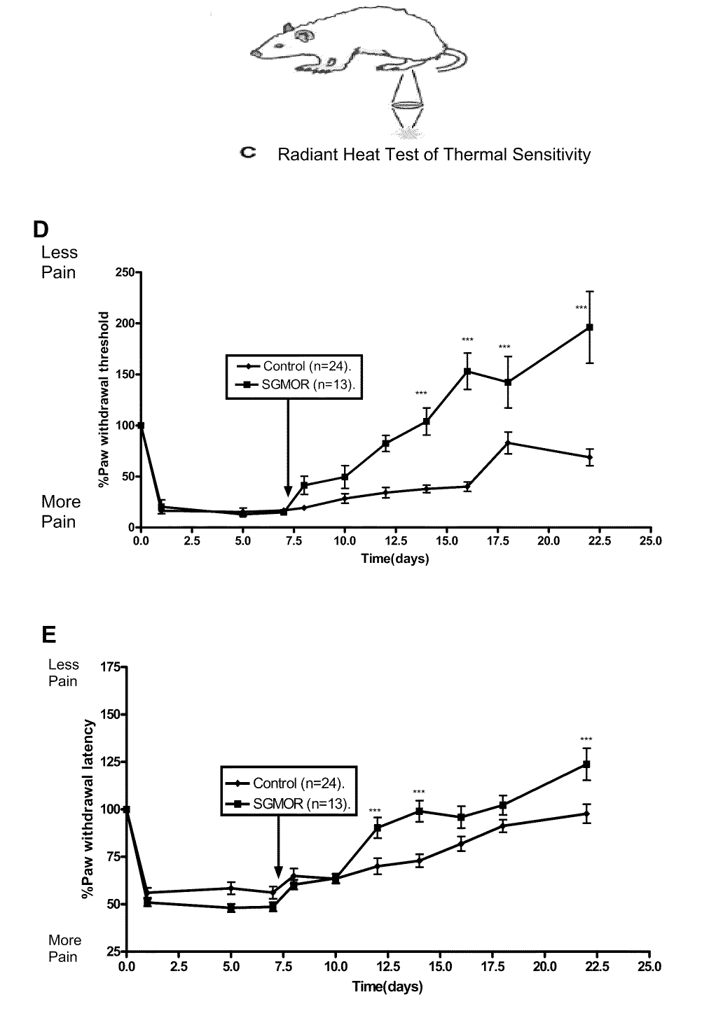

In rodent models of acute phasic pain, infection with this virus increased pain thresholds to thermal and mechanical stimuli (FIG. 1). In a rodent model of acute inflammatory pain using capsaicin (the “hot” in hot chili peppers), infection with this virus reduces spontaneous pain-associated behaviors and evoked pain responses to thermal stimuli (FIG. 2). Herpes simplex viral vectors can be used to modulate expression of delta opioid receptors for the treatment of neuropathic pain-associated behavior and to enhance opioid analgesia, as illustrated in a mouse L5 spinal nerve transection model. On day 7 post-nerve transection mechanical allodynia and thermal hyperalgesia was verified and a recombinant herpes simplex virus type 1 encoding the cDNA sequences for the sense mu opioid receptor (SGMOR), or E. coli LacZ gene (control) was administered to the hindpaw. Infection with the SGMOR virus both reverses neuropathic pain-associated behaviors and enhances opioid analgesia (FIG. 3).

When peripheral nerves are damaged the expected sensory loss is often accompanied by mild to severe pain. This pain is termed neuropathic because it is believed to be due to an injury and dysfunction of the nervous system. Neuropathic pain is often chronic in nature and resistant to the current therapies. Several types of painful peripheral neuropathies (the dysfunction lies in the peripheral nervous system) are observed in the clinic. The current theory is that they all share at least some underlying pathogenic mechanisms. Post-traumatic painful peripheral neuropathy is used to distinguish pain that arises from damage to the nerves by penetrating wounds, crush (includes crush due to tumors or low back pain as a result of disk herniation), stretch, and surgery versus pain as a result of nerve injury due to disease such as postherpetic neuralgia and diabetic neuropathy, or pain as a result of chemical agents such as anti-neoplastic agents and alcohol.

The use of animal models has had an exponential impact on understanding the mechanisms related to chronic neuropathic pain states. More importantly, these models have been used in pre-clinical drug development for agents that may treat neuropathic pain. An increased understanding of the pathophysiological mechanisms of neuropathic pain has led to the discovery of several new classes of drugs to target neuropathic pain. Of interest many of these new drugs are not traditional analgesic agents in that they do not suppress normal acute (physiological pain) pain sensation but rather only alleviate neuropathic pain (pathological pain). These agents would have been impossible to discover using animal models that were traditionally being used a decade ago for the discovery and development of pain therapeutics. These newer agents discovered in rodent models of neuropathic pain are better termed anti-allodynics and anti-hyperalgesics rather than analgesics.

There are several well described models of painful peripheral neuropathy due to traumatic or partial nerve damage. The number of models is increasing yearly. The three most well known models are the chronic constriction injury of Bennett and Xie (Pain, 33,87, 1988), the partial nerve transection model of Seltzer (Pain, 43,205, 1990), and the spinal nerve transection injury of Kim and Chung (Pain, 50,355, 1992), all of which are incorporated herein by reference. Although the mode of producing the injury is different across the models the abnormal stimulus-evoked pain is similar. This pain is characterized by thermal hyperalgesia and mechanical allodynia and hyperalgesia. Hyperalgesia and allodynia are reported in chronic neuropathic pain patients. Signs of spontaneous or ongoing pain (limping and guarding of the affected hindpaw) can also be observed. The three models have been compared directly in terms of their behavioral outcomes (Kim, Yoon, Chung, Exptl Brain Res. 113, 200, 1997). The maximum severity of pain is very similar but onset and duration of behaviors varies between the models.

The original spinal nerve transection model described by Kim and Chung involved tight ligation (and hence transection) of the L5 and L6 spinal nerves close to their respective ganglia. This results in a partial deafferentation of the nerves that have axons traveling in the L5 and L6 roots (sciatic and saphenous). As a result, the hind paw is innervated by approximately 50% fewer afferent fibers (all types). A modification of the Chung model was utilized herein to directly transect only the L5 spinal nerve. This has been found to produce equivalent behaviors with greater reproducibility and reliability. Close to 100% of animals go on to develop allodynia and hyperalgesia. Allodynia and hyperalgesia are present for 1-2 months after injury.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention that is defined in the following claims and equivalents thereto. Further, it is recognized that many embodiments may be conceived that do not achieve all of the advantages of some embodiments, yet the absence of a particular advantage shall not be construed to necessarily mean that such an embodiment is outside the scope of the present invention.

What is claimed:

1. An expression cassette comprising a heterologous nucleotide sequence that encodes a mu opioid receptor.

2. An expression cassette as in claim 1, further comprising regulatory sequences operably linked to the nucleotide sequence, the regulatory sequences including a translation leader sequence, a promoter, and a transcription termination sequence.

3. An expression cassette as in claim 2, wherein the promoter is heterologous to the coding sequence.

4. An expression cassette as in claim 2, wherein the promoter is an inducible promoter.

5. A vector comprising the expression cassette of claim 1.

6. A vector as in claim 5, wherein the vector is a viral vector.

7. A vector as in claim 6, wherein the viral vector comprises a herpes viral vector.

8. A transformed cell comprising the vector of claim 5.

9. A transformed cell as in claim 8, wherein the cell is a nervous system cell.

10. A transformed cell as in claim 8, wherein the cell is a nerve cell.

11. A method for over-expression of a mu opioid receptor in a cell comprising introducing an expression cassette into the cell in an amount sufficient to over-express the mu opioid receptor, the expression cassette comprising a heterologous nucleotide sequence that encodes a mu opioid receptor, wherein the expression cassette over-expresses the mu opioid receptor.

12. A method as in claim 11, wherein the expression cassette further comprises regulatory sequences operably linked to the nucleotide sequence, the regulatory sequences including a translation leader sequence, a promoter, and a transcription termination sequence.

13. A method as in claim 12, wherein the promoter is heterologous to the coding sequence.

14. A method as in claim 12, wherein the promoter is an inducible promoter.

15. A method as in claim 11, wherein the expression cassette is introduced via a vector.

16. A method as in claim 15, wherein the vector is a viral vector.

17. A method as in claim 16, wherein the viral vector comprises a herpes viral vector.

18. A method as in claim 11, wherein the cell is a nervous system cell.

19. A method as in claim 11, wherein the cell is a nerve cell.

20. A non-human mammal comprising the expression cassette of claim 1.
